(19) United States
(12) Patent Application Publication
(10) Pub. No.: US 2005/0021070 A1
(43) Pub. Date: Jan. 27, 2005

(54) Methods and apparatus for manipulating vascular prostheses

(75) Inventors: Tanhum Feld, Moshav Merhavya (IL); Eitan Konstantino, Orinda, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AngioScore, Inc., Alameda, CA (US)

(21) Appl. No.: 10/917,902

(22) Filed: Aug. 13, 2004

Related U.S. Application Data
(63) Continuation-in-part of application No. 10/810,330, filed on Mar. 25, 2004.
Application No. 10/810,330 is a continuation-in-part of application No. 10/631,499, filed on Jul. 30, 2003.
(60) Provisional application No. 60/442,161, filed on Jan. 21, 2003.

(51) Int. Cl. A61F 2/06

(52) U.S. Cl. 606/194

(57) ABSTRACT

An interface structure is provided over a catheter having an inflatable balloon or other expansible structure for deploying or manipulating a stent. The interface structure is typically a cage having a plurality of helical interface elements which are disposed between the balloon and the stent during the expansion or manipulation. Use of the interface structure promotes uniform expansion and deformation of the stent.

### CROSS-REFERENCES TO RELATED APPLICATIONS

This application is a continuation-in-part of commonly assigned, co-pending U.S. application Ser. No. 10/810,330, filed on Mar. 25, 2004 (Attorney Docket No. 021770-000120US), which is a continuation-in-part of U.S. application Ser. No. 10/631,499, filed on Jul. 30, 2003 (Attorney Docket No. 021770-00010US), which claims the benefit under 35 USC §119(e) of U.S. Provisional Application No. 60/442,161, filed on Jan. 21, 2003 (Attorney Docket No. 021770-000100US), the full disclosures of which are incorporated herein by reference.

### BACKGROUND OF THE INVENTION

1. Field of the Invention

The present invention relates generally to medical methods and apparatus and more particularly to the delivery and manipulation of stents and other prostheses in the vascular system.

Balloon dilatation (angioplasty) is a common medical procedure mainly directed at revascularization of stenotic vessels by inserting a catheter having a dilatation balloon through the vascular system. The balloon is inflated inside a stenosed region in a blood vessel in order to apply radial pressure to the inner wall of the vessel and widen the stenosed region to enable better blood flow.

In many cases, the balloon dilatation procedure is immediately followed by a stenting procedure where a stent is placed to maintain vessel patency following the angioplasty. Failure of the angioplasty balloon to properly widen the stenotic vessel, however, may result in improper positioning of the stent in the blood vessel. If a drug-eluting stent is used, its effectiveness may be impaired by such improper positioning and the resulting restenosis rate may be higher. This is a result of several factors, including the presence of gaps between the stent and the vessel wall, calcified areas that were not treated properly by the balloon, and others.

Stent placement can be particularly difficult when the plaque material is hard, fibrotic, or calcified and interferes with the uniformity of stent expansion. Balloon inflation of the stent occurs preferentially in the softer or least resistant areas of the stenotic material. Using high balloon inflation pressure to expand the stent in the more resistant regions can often cause stretching and damage to the vessel wall in the regions of softer stenotic material.

Stent placement is also problematic when the treated region is at a blood vessel bifurcation. When a stent is placed in a main vessel, the opening to a side branch can be covered or “jailed” by the stent struts. Such interference with the opening to the side branch is particularly troublesome when it is necessary to enter the side branch for further treatment. In such cases, a balloon catheter is typically used to open a cell in the stent to minimize interference.

The use of conventional angioplasty balloons to open “holes” in the side of a stent can be quite difficult. If the stent struts are broken, they may damage the blood vessel wall and/or the balloon. When a conventional angioplasty balloon is used to open a cell of a stent, the balloon will first expand at the distal and the proximal areas of the balloon. Expansion of the center of the balloon will usually be constrained by the cell until the cell resistance is abruptly overcome. The cell will then rapidly expand in an uncontrolled manner when the internal pressure overcomes the cell resistance. Even when the stent remains intact, opening of the cell can be non-uniform, leaving an irregular passage for subsequent introduction of the angioplasty catheter needed to treat the side branch.

For these reasons, it would be desirable to provide improved balloon and other catheters for the delivery and manipulation of stents and other vascular prostheses. In particular, it would be desirable to provide delivery methods and apparatus which are capable of delivering and opening a prosthesis in a highly uniform manner, regardless of the degree of calcification which may be present in the plaque or other stenotic material being treated. It would be further desirable to provide stent delivery structures which are able to uniformly apply relatively large expansion forces to the interior of the stent or other prostheses being opened. It would still further be desirable to provide improved methods and apparatus for opening passages in the stent or other prosthesis after it has been delivered. Such apparatus and methods should provide for uniform and effective opening of the interior of a cell of the stent, particularly to provide passage into a side branch vessel covered by the stent. At least some of these objectives will be met by the inventions described hereinafter.

2. Description of the Background Art

U.S. Pat. No. 6,129,706 and U.S. Published Application 2003/0032973 describe balloons having spiral or other surface structures which may be used for delivering prostheses. U.S. Pat. No. 6,245,040 and U.S. Published Applications 2003/0153870 and 2004/0111108 describe structures placed over dilatation balloons for various purposes including perfusion, anti-slip, and plaque cutting. Other modified balloon structures having helical geometrics are described in U.S. Pat. Nos. 5,545,132 and 5,735,816; and U.S. Published Application 2003/0144683. U.S. Pat. No. 6,447,501 describes a stent delivery system with a guidewire extending over the stent expansion balloon.

### BRIEF SUMMARY OF THE INVENTION

The present invention provides improved methods and apparatus for the delivery and manipulation of stents and other prostheses in the vasculature and other body lumens. In a first aspect, the present invention is particularly intended for the delivery of vascular prostheses within regions of fibrotic, calcified, or otherwise hardened plaque or other stenotic material of the type which can interfere with stent expansion using conventional angioplasty balloons. In a second particular aspect, the present invention will be useful for opening passages through the wall of a previously implanted stent or other vascular prosthesis. Usually, the opening will be into a branch vessel through a prosthesis in the main vessel which at least partly covers or blocks the opening or os to the branch vessel. The methods and apparatus will find their greatest use in the treatment of the arterial vasculature, including but not limited to the coronary arterial vasculature, but may also find use in the treatment of the venous and/or peripheral vasculature, and in the delivery of other prostheses to other body lumens outside of the vasculature.

In a first aspect of the present invention, a method for expanding a vascular prosthesis within a region of hardened plaque comprises delivering a prosthesis to the region of hardened plaque. A shell is then expanded within the prosthesis to cause expansion, where the shell is disposed within an interface structure which engages an inner surface of the prosthesis as it is expanded. The interface surface is adapted to engage the inner surface of the prosthesis without causing damage and in a manner which provides a number of expansion points to promote uniform expansion of the prosthesis.

According to the second aspect of the present invention, a passage through the wall of the prosthesis in a main vessel is opened by positioning an expansible shell, typically an inflatable balloon, through a cell in the prosthesis. An interface structure surrounds the expansible shell, and the interface structure engages the periphery of the cell as the shell is expanded within the cell to open the cell.

As is well known in the art, stents and other vascular prostheses include expansible cells which expand when the prosthesis is radially opened within the target blood vessel. The cells may be “open” or “closed”. Open cells are characteristic of conventional serpentine and zig-zagged stent structures. Closed cells, in contrast, are characterized by relatively small, closed rectangular, diamond, or other structures with a closed periphery. The present invention will be suitable for expanding an open region or a passage through either type of cell by expanding the shell therein. Particularly, by providing the interface structure, the balloon can apply relatively uniform or equal forces at a number of points on the shell in order to promote uniform opening and limit possible damage to the balloon or other shell from the stent.

Both aspects of the method of the present invention may employ a similar interface structure which comprises a plurality of interface elements each having an outwardly exposed surface which engages either the inner surface of the prosthesis or an inner circumference of a cell of the prosthesis. The outwardly exposed surface is preferably free from scoring features (in contrast to the earlier applications of the assignee of the present application) which could damage the prosthesis. For example, the outwardly exposed surfaces may be flattened and have rounded corners. The flattened surface will provide an efficient transfer of outward force, while the rounded corners will prevent scoring or damage to the stent or other prosthesis. Usually, the interface structure will comprise a plurality of such interface elements, and the interface elements will be arranged helically over the expansion balloon or other expansible shell. Usually, the interface structure will be elastic, e.g. being composed from a superelastic material, so that it will close the shell after expansion is completed.

The present invention still further provides a stent manipulation catheter which is useful for performing these methods. The stent manipulation catheter comprises a catheter body having a proximal end and a distal end. A radially expansible shell is disposed near the distal end of the catheter body, and the interface structure circumscribes but is not attached to the shell. Usually, the interface structure comprises at least one continuous interface element extending over the entire length of the shell, typically being arranged helically over the shell. The interface structure will usually comprise two, three, four or more individual interface elements, typically all being arranged helically. The total exposed area of the shell, however, will be below 20% of the expansible area of the shell, preferably being below 10%, and usually being below 5%. In the exemplary cases, the interface structure may comprise a wire, a chemically etched strut, or the like.

The interface structure is preferably incorporated into a cage structure which circumscribes the expansible shell. The cage structure is preferably unattached to the expansible shell but usually attached at at least one point to the catheter body. In a specific embodiment, the cage structure is attached to the catheter body by an attachment structure having a proximal end attached to the catheter body and a distal end attached to the cage structure. The attachment structure is sufficiently sized and compliant to accommodate geometrical and reaction forces produced by the cage structure as it is expanded by the shell. Further preferably, the assembly of the shell and the interface structure will be sufficiently flexible to permit it to bend at a radius of 10 mm or less as the catheter is advanced through the coronary or other vasculature.

### BRIEF DESCRIPTION OF THE DRAWINGS

FIG. 1A illustrates a catheter constructed in accordance with the principles of the present invention, where an attachment structure joins the interface structure to the catheter body.

FIG. 1B illustrates the structure of FIG. 1A shown without the balloon.

FIGS. 2A-2C illustrate a catheter constructed in accordance with the principles of the present invention having an attachment structure with various patterned perforations.

FIG. 3 illustrates another embodiment of a catheter constructed in accordance with the principles of the present invention having a tapered attachment structure.

FIG. 4 illustrates yet another alternative embodiment of a catheter constructed in accordance with the principles of the present invention, where an attachment structure is connected to a manipulator.

FIG. 5 illustrates an embodiment of the invention having a laminated section at the distal end of the compliance tube.

FIG. 6 illustrates another view of the embodiment of FIG. 5.

FIGS. 7A and 7B are alternative cross-sectional views taken along line 7 of FIG. 6.

FIG. 8 illustrates the embodiment of FIG. 5 with an expandable balloon inserted within the scoring structure.

FIG. 9 illustrates an embodiment with a sleeve over the distal end of the interface structure.

FIG. 10 illustrates a method of the present invention utilizing an insertion tube to mount the interface structure over the expandable balloon.

FIG. 11 illustrates shows the insertion tube inserted over the expandable balloon.

FIG. 12 illustrates a scoring catheter of the present invention with the insertion tube removed.

FIGS. 13A-13D illustrate a method for expanding a prosthesis cell aligned with the opening of a side branch vessel in accordance with the principles of the present invention.

FIGS. 14A and 14B compare the use of a conventional angioplasty balloon for expanding a stent cell and use of the stent interface structure of the present invention for expanding a stent cell.

FIGS. 15A and 15B illustrate use of the stent interface structure of the present invention for expanding a stent in a blood vessel.

### DETAILED DESCRIPTION OF THE INVENTION

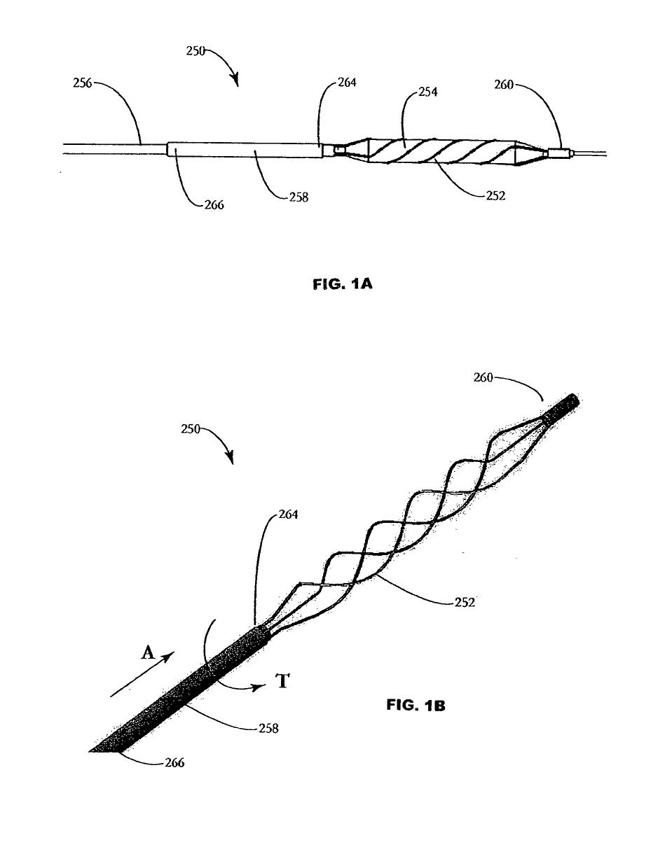

Referring now to FIGS. 1A and 1B, an angioplasty catheter 250 having an axially distensible attachment structure 258 is illustrated. An interface structure 252 is held over an expandable shell, typically a dilatation balloon 254, and is fixed at one end to the distal end 260 of catheter body 256. Proximal end 262 of interface structure 252 is connected to the distal end 264 of attachment structure 258. The proximal end 266 of attachment structure 258 is fixed to the catheter body 256. As described below, the attachment structure 258 may be configured to reduce forces applied on the external structure 252 and the catheter body 256 during expansion and contraction of balloon 254.

The interface structure 252 is illustrated as three separate helical interface elements, typically composed of nitinol or other superelastic material. While this is a presently preferred geometry, it will be appreciated that the number of interface elements may vary from one to ten or even greater. Moreover, while the helical geometry is preferred, it is not essential and the interface elements could be straight, serpentine, zig-zag, or have any one of a variety of other configurations which permit expansion of the balloon therein. The helical structure is generally preferred, however, since it reduces the risk of the elements interfering with the stent structure as the balloon is used to expand a stent or other prosthesis and/or pass through a cell of the stent structure in order to permit subsequent expansion.

Attachment structure 258 typically comprises a cylindrical over-tube, or compliance tube, made of an elastic material. Over-tube 258 generally has an inner diameter that is slightly greater than the outer diameter of the catheter body 256. Because only a small section of the proximal end of the attachment structure 258 is fixed to the catheter body, the distal end 264 attached to interface structure 252 is free floating, and is free to slide axially and rotationally with respect to catheter body 256. Attachment structure 252 may be fixed, for example by adhesion, directly to the to catheter body 256 and external structure 252, or to a collar or other intermediate attachment means.

As balloon 254 is expanded, interface structure 252 expands in circumference and contracts axially along the catheter body 256, creating axial force in the direction of arrow A on attachment structure 258. Attachment structure 258, fixed to the catheter at its end 266, axially stretches to accommodate the axial movement of the interface structure 252. Interface structure 252 also tends to rotate about the catheter body 256, causing a torsional force T. The distal end 264 of attachment structure 258 rotates through the full range of motion of scoring structure 252 to accommodate torsional force T, while proximal end 266 remains stationary with respect to catheter body 256.

The configuration illustrated in FIGS. 1A and 1B allows the compliance of the expandable system to be controlled. Generally, where one end of the scoring structure is free, the compliance of the expandable system will be a combination of the compliance of the balloon and the scoring structure. However, because the ends of the expandable system shown in FIGS. 1A and 1B are fixed at distal end 260 and proximal end 266, the attachment structure controls the compliance of the expandable system.

The compliance of the system may be varied by any combination of material selection, wall thickness, or length of the over-tube 258. Over-tube 258 may comprise any elastomer, such as elastic polymer like Nylon, Pebax, or PET. Typically, compliance tube 258 is formed from extruded tubing, but is may also comprise braided polymeric or metallic fibers, or wire mesh. A superelastic metal such as nitinol or stainless steel may also be used. Where the compliance tube comprises an extruded polymeric tube, the wall thickness can vary in the ranges set forth above, and the length of the tube can range from 1 cm to 10 cm. For the same material, the thinner-walled and longer the tube, the more compliant the system.

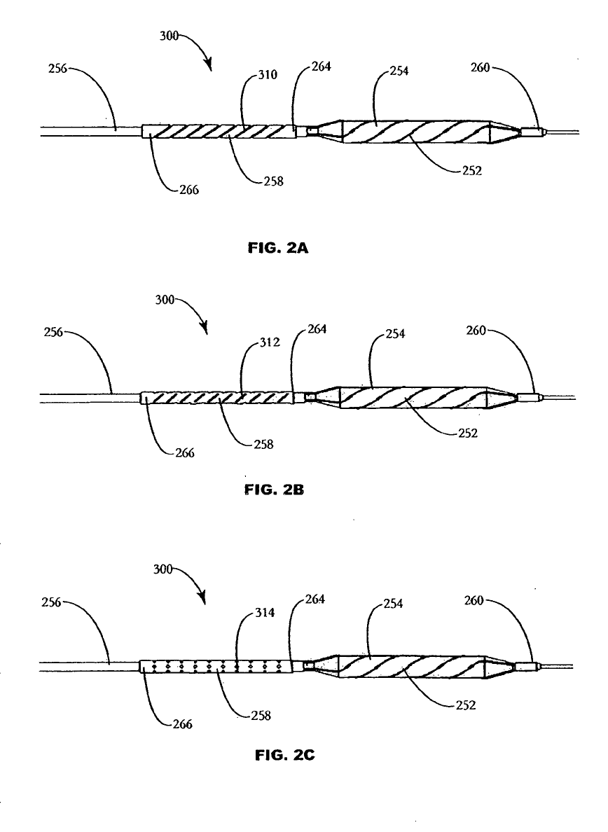

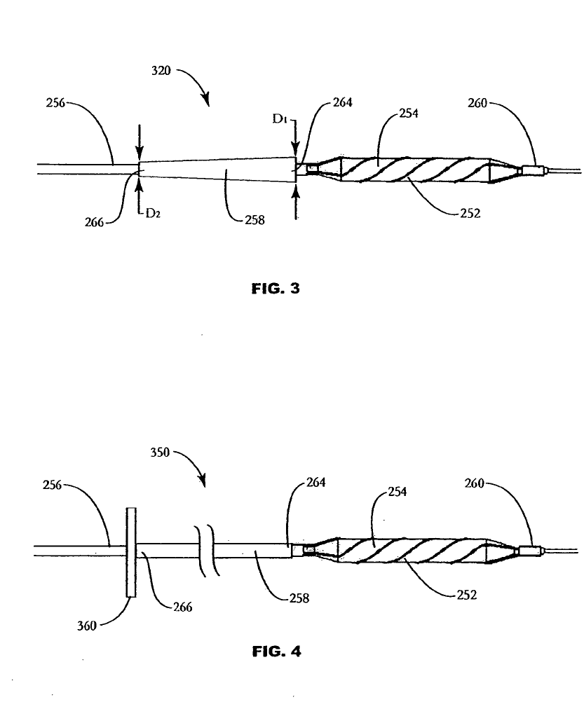

Referring to FIGS. 2A to 2C, the axial and rotational compliance of the compliance tube 258 may also be varied by creating one or more perforations in compliance tube 258. The perforations may comprise one or more slots in the circumference of the tube. The slots may comprise one continuous slot spiraling across the length of compliance tube 258, or may be a number of slots aligned in any number of patterns, such as helical 312, or radial 314. The slots may also be any number of shapes, such as circular or rectangular, and may have a discreet length or be contiguous across the surface of the compliance tube.

Referring to FIG. 3, the outside diameter of compliance tube 258 may be tapered to facilitate delivery and retrieval of the scoring catheter 320 from the treatment site within the lumen. Generally, the outer diameter will be larger at the distal end 264 of the compliance tube 258 and smaller at the proximal end 266 of the compliance tube. The outside diameter D1 at the distal end will vary depending on the profile of the scoring structure and balloon when collapsed but typically range from 0.004 in. to 0.01 in. larger than the outside diameter D2 at the proximal end. The outside diameter D2 at the proximal end is generally as close as possible to the outside diameter of the catheter body to create a smooth transition between the compliance tube and the catheter. As an example, for a catheter body having an outside diameter of 0.033 in., outside diameter D1 at the distal end may be 0.042 in. with an inner diameter of 0.038 in., the inner diameter providing clearance between the catheter body so that the distal end of the compliance tube can move relative to the catheter body. Correspondingly, the outside diameter D2 at the proximal end may taper down to 0.0345 in., with an inner diameter of 0.034 in. to closely match the catheter body having outside diameter with enough clearance to be bonded to the catheter body by an adhesive.

The taper may run across the whole length of the compliance tube, or alternatively be only tapered at a section of the length of the compliance tube. The tapered compliance tube 258 smoothes the transition between the scoring structure and catheter body, and minimizes the likelihood of the outer tube or scoring structure snagging or catching on a portion of the luminal wall during delivery or retrieval of the catheter.

Now referring to FIG. 4, an alternative embodiment of a stent manipulation catheter 350 is shown having a manipulator 360. The attachment structure 258 is connected at its distal end 264 to the scoring structure 252. Instead of being secured directly to the catheter body 256, the proximal end 266 is attached to manipulator 360. Typically, the manipulator 360 is positioned at the proximal end of the catheter body 256 and the attachment structure 258 extends from the interface structure across the length of the catheter body. Like the above embodiments, the attachment structure is capable of axially and rotationally extending to accommodate foreshortening of the interface structure as the shell is expanded.

In some embodiments, the compliance of the interface structure 252 and balloon 254 is controlled by actuating the manipulator during expansion or contraction of the radially expandable shell. In one aspect, the attachment structure 258 may be axially advanced with respect to the catheter body 256 as the balloon is being inflated or deflated. For example, the attachment structure 258 may be pulled away from the distal end of the catheter body 256 while the balloon 254 is being expanded to constrain the compliance of balloon. The attachment structure 258 may also be pulled away from the distal end of the catheter body 256 during or after the balloon 254 is being deflated to minimize the profile of the balloon and scoring structure. Alternatively, the manipulator 360 may be used to rotate the attachment structure 258 with respect to the catheter body 256 to control the compliance of the balloon and scoring structure during transition from a collapsed to expanded state and back to a collapsed state.

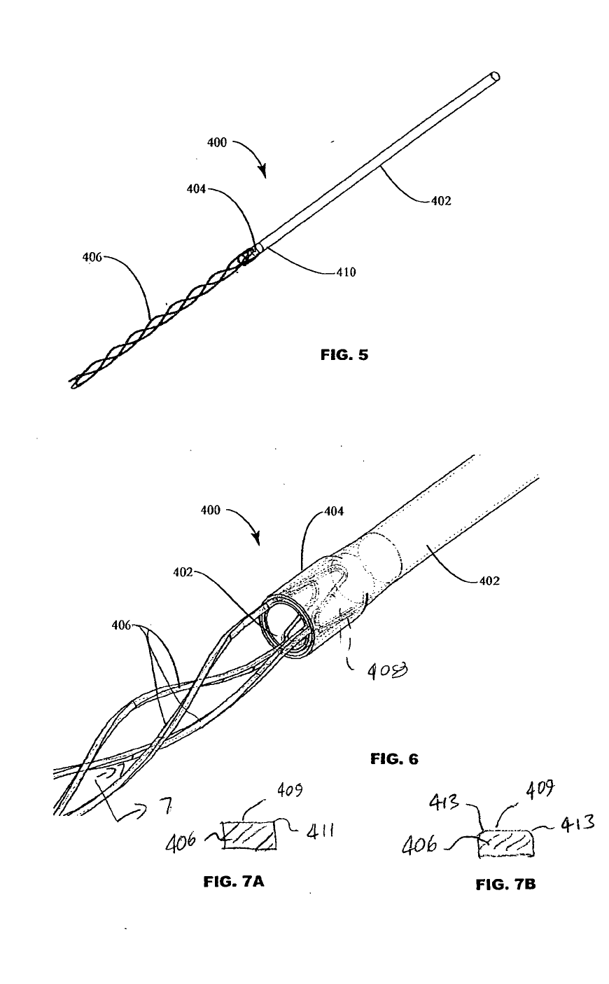

Now referring to FIGS. 5 and 6, an interface cage structure 400 is illustrated having a two-layer laminated compliance tube 402. As shown in FIG. 22, the compliance tube 402 has a laminated structure 404 at at least its distal end 410. The laminated structure holds the proximal ends 408 of the interface elements 406 as shown in broken line in FIG. 22. The interface elements 406 may be sized to fit over the outside of the compliance tube 402, as illustrated in FIG. 22, with the lamination covering the elements. Alternatively, the compliance sleeve tube 402 may be sized to fit inside of the interface structure 406, with the laminating layer(s) formed over the elements 406 (not shown).

The laminating structure may be composed of a polymer similar to the compliance tube 402, and may be heat shrunk or melted to thermally bond the compliance sleeve to the compliance tube and sandwich the interface elements 406. Alternatively, an adhesive or other bonding method such as ultrasonic or RF energy may be used to laminate the structure. The laminated structure as shown in FIGS. 5 and 6, provides a smoothed transition and strengthened bond between the scoring cage and the attachment structure. Such a smooth transition is a particular advantage when withdrawing the scoring cage from the vasculature.

The interface elements 406 are shown to have a generally square or rectangular cross section. For example, the elements 406 may have a rectangular cross section as shown in FIG. 7. This cross section includes a flat top which is the region which will engage the stent or other prosthesis as the cage is expanded therein. This cross section, however, has relatively sharp corners 411. Such sharp corners present a risk of damaging the stent when the cage is expanded therein. Thus, it will often be preferred to utilize interface element 406 as illustrated in FIG. 7B where the flat surface 409 is located between rounded corners 413. While the flat surface 409 is generally preferred since it distributes force evenly to the stent, it would of course be possible to provide a slight bending or crown to the surface while still delivering the uniform force. It will generally be undesirable, however, to employ structures which impart a concentrated force as is generally desirable for use in “cutting balloons” and other angioplasty devices intended to score plaque upon inflation.

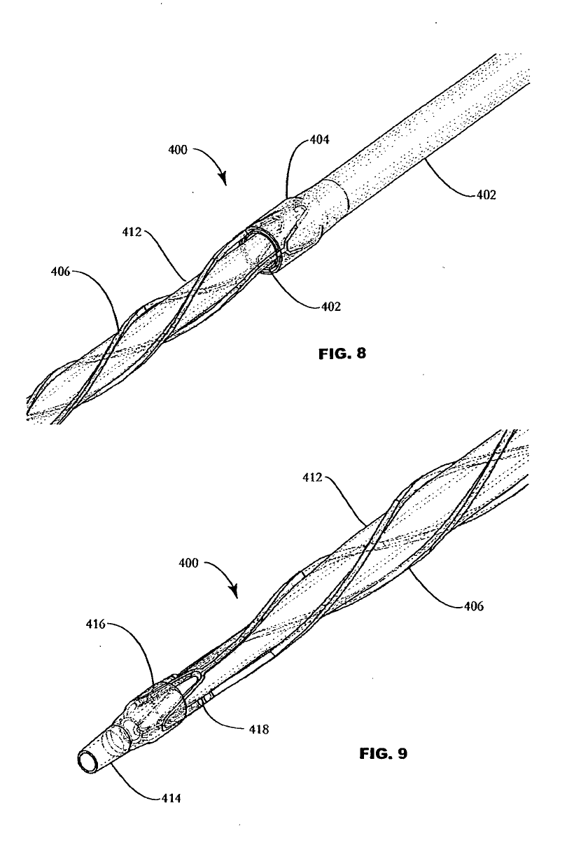

FIGS. 8 and 9 illustrate interface cage 400 positioned over an expandable dilation balloon 412. As shown in FIG. 24, distal end 418, of the interface cage may be coupled to the distal tip 414 of the catheter body by an end cap 416. The end cap 416 may be composed of a compatible polymer and thermally bonded with the catheter body to fix distal end 418 of the interface structure to the catheter body.

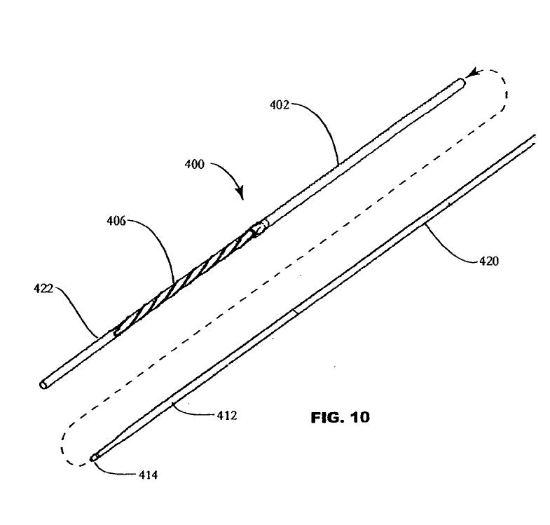

Now referring to FIGS. 10 to 12, a method is illustrated for mounting an expandable interface cage 406 over a balloon catheter. The interface cage 406 is pre-expanded by loading it over an insertion tube 422 that has an inner diameter slightly larger than the outer diameter of the balloon 412. A catheter body 420 having a balloon 412 is then inserted into the inner diameter of the insertion tube 422 and advanced until the balloon 412 is appropriately positioned with respect to the interface structure 406, as illustrated in FIG. 11. The insertion tube 422 is then pulled back to allow the expanded scoring structure to collapse over the balloon 412 and the catheter body 420, as shown in FIG. 12. The interface structure 406 may then be secured at its distal end 418 to the distal tip 414 of the catheter body 420 and the proximal end 424 of the interface structure/attachment structure assembly to a medial location on the catheter body 420.

Referring now to FIGS. 13A-13D, use of a balloon catheter 500 for expanding the interior periphery of a cell C and a stent S is described. The stent S has been placed in a main vessel Mv having a branch vessel V creating a bifurcation. The catheter 500 carries an interface structure 510 over an expansible balloon 512 or other shell structure. The catheter is guided through the main vessel Mv lumen and through the interior of the cell C, typically over a guidewire GW. The interface structure 510 is positioned so that it is centered within the cell, typically by viewing the position fluoroscopically during the procedure. Once the interface structure 510 is properly positioned, the balloon 512 may be inflated, as shown in FIG. 13C. By applying the proper expansion force, typically a pressure in the range from 4 atmospheres to 20 atmospheres, the cell may be uniformly expanded, as illustrated in FIG. 13D.

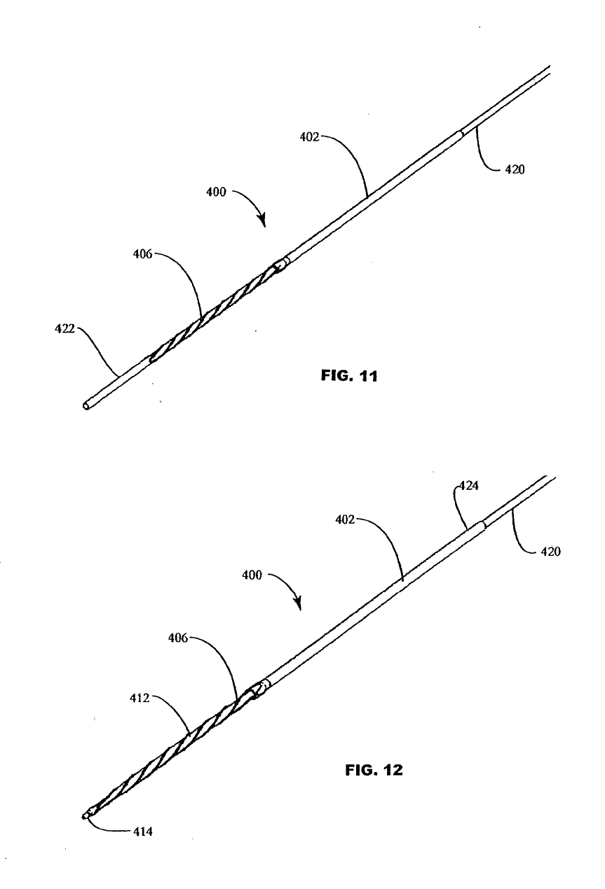

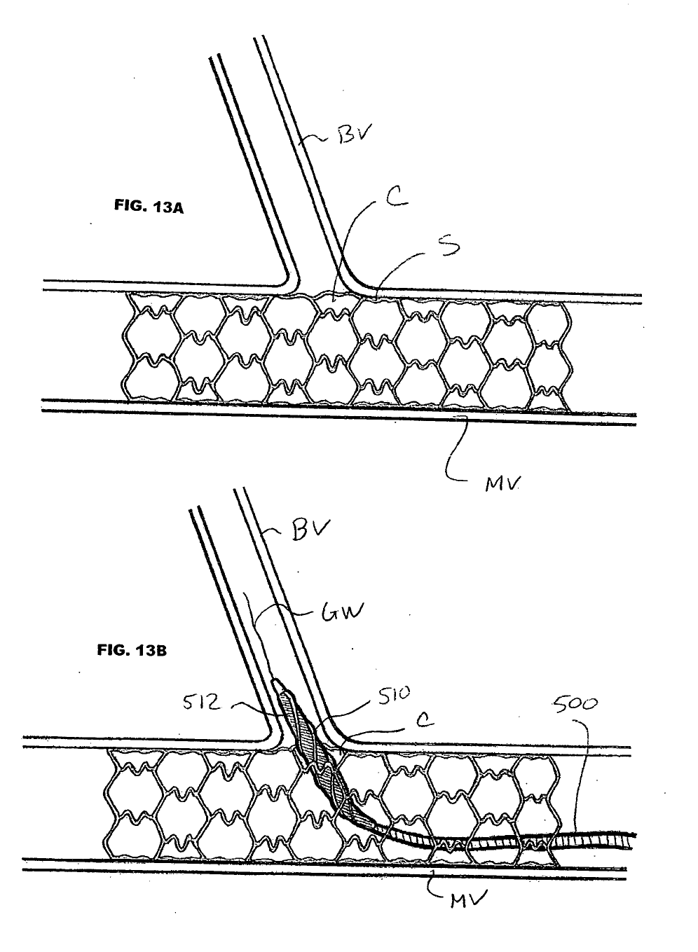

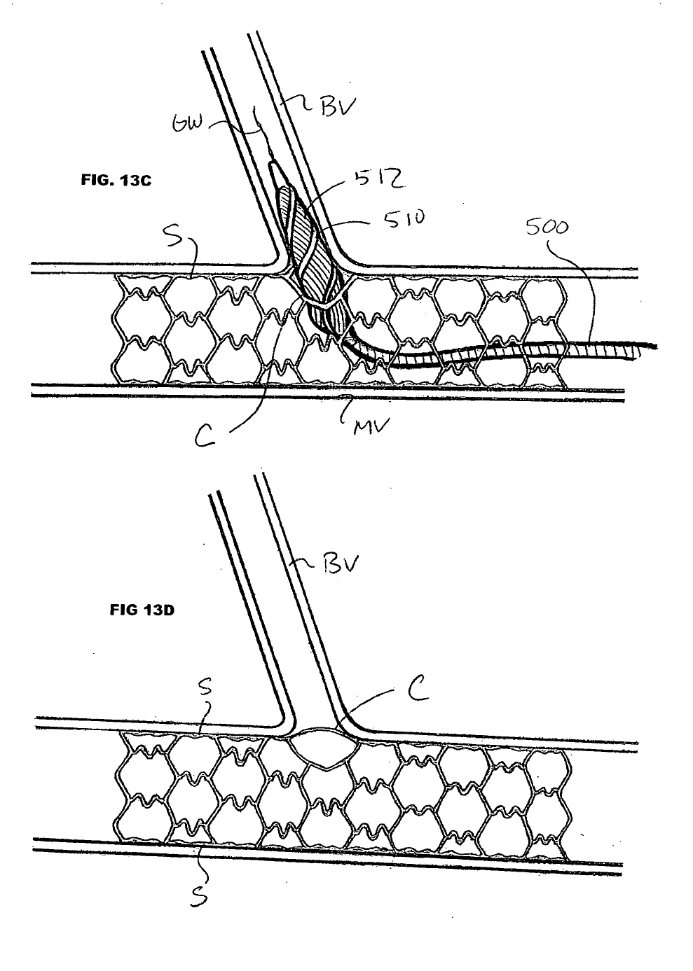

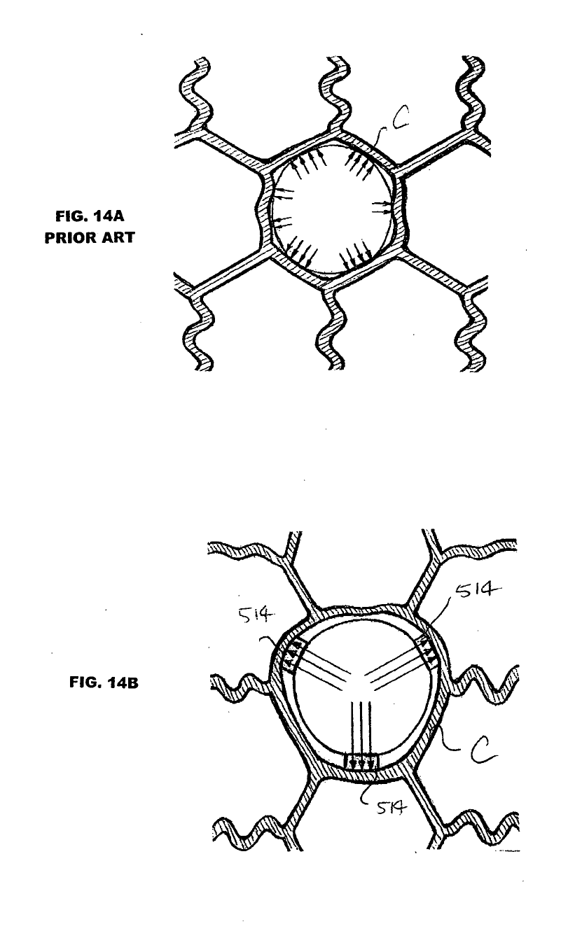

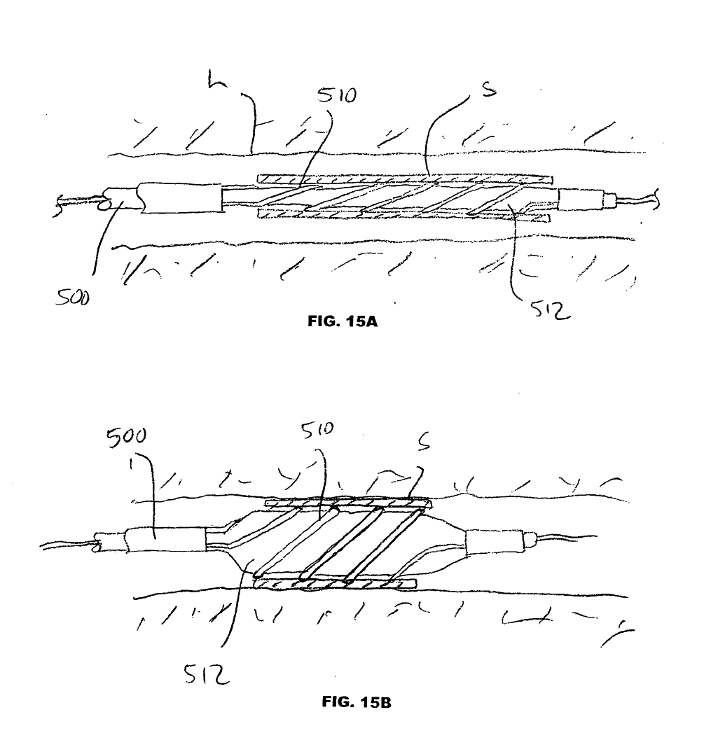

Referring now to FIGS. 14A and 14B, the advantage of using the interface structure 510 will be described. Shown in FIG. 14A, use of a conventional angioplasty balloon without an interface structure results in a generally uneven expansion force at different points about the periphery of cell C. In particular, where the balloon is able to contact a greater length of the cell, a higher force will be applied. In contrast, use of the individual elements 514 of the interface structure 510 will provide a very uniform expansion force at the points where the periphery of the cell C is engaged. Another advantage is that the cage prevents the balloon from necking within the stent cell and thus can avoid the abrupt opening which can be experienced with the use of conventional balloons and can create a more uniform and controlled expansion of stent cell. Such controlled linear expansion is much less likely to cause damage the stent struts and as a result to the balloon and the blood vessel.

Referring now to FIGS. 15A and 15B, in another embodiment of the invention, the catheter 500 carries a stent S or other vascular prosthesis. The stent S is typically crimped over the interface structure 510, which is typically a helical unit. In this way, the interface structure 510 can push the stent against hard areas of the lesion L, enabling proper positioning of the stent against the vessel wall, even in hard or calcified lesions and without pre-dilation, as shown in FIG. 15B.

Using the balloon or other expandable shell expandable shell with the interface structure to deliver stents enables the transmission of larger forces to the lesion through stent to the surrounding vessel wall and enhances better wall apposition of the stent even in hard lesions. In many cases stents have poor wall apposition in lesions with non uniform calcification. In those lesions the balloon yields at the calcified segments and the stent does not fully deploy in such segments. By using the interface surface of the present invention, the balloon or other expandable shell uniformly distributes the outward forces and supports the stent during expansion and allows full dilatation even in calcified segments. This advantage is even more important with thin wall stents which individual cells with lower radial force since the struts are very thin in comparison to conventional stents. The use of the interface structures of the present invention should in at least some instances reduce or eliminate the need for pre dilatation.

Additionally, when the balloon is deflated after the stent has been deployed, the interface structure helps deflate the balloon by applying an inward radial force which helps prevent the balloon from “winging.” Winging occurs when the balloon deflates to a flat shape. The flat balloons is very narrow in one axis but wider than the vessel in the other axis. The balloon can thus have a tendency to get caught by a stent strut or rub against the vessel wall, making the balloon retrieval difficult. At worst, balloon capture by a stent strut can cause a failure of the procedure enhanced by the interface surface can result in a low profile deflated balloon which is easier to remove.

It will be appreciated by persons skilled in the art that the present invention is not limited to what has been particularly shown and described hereinabove. Alternate embodiments are contemplated that fall within the scope of the invention.

1. A method for opening a passage into a branch vessel through a prosthesis in a main vessel, said method comprising:
positioning an expansible shell through a cell in the prosthesis, wherein an interface structure surrounds the expansible shell; and
expanding the shell to expand the structure within the cell to open the cell and create the passage.

2. A method as in claim 1, wherein the interface structure comprises a plurality of interface elements each having an outwardly exposed surface which engages an inner circumference of the cell.

3. A method as in claim 2, wherein the outwardly exposed surface is free from scoring features.

4. A method as in claim 3, wherein the outwardly exposed surface is flattened with rounded corners.

5. A method as in any one of claims 2 to 4, wherein the interface structure comprises a plurality of interface elements.

6. A method as in claim 5, wherein the interface elements are arranged helically over the shell.

7. A method as in any one of claims 2 to 4, wherein the interface structure is elastic so that it closes the shell after expansion.

8. A method for expanding a vascular prosthesis within a region of hardened plaque, said method comprising:
delivering the prosthesis to the region of hardened plaque; and
expanding a shell within the prosthesis to cause expansion, wherein the shell is disposed within an interface structure which engages an inner surface of the prosthesis as it is expanded.

9. A method as in claim 8, wherein the interface structure comprises a plurality of interface elements each having an outwardly exposed surface which engages the inner surface of the prosthesis.

10. A method as in claim 9, wherein the outwardly exposed surface is free from scoring features.

11. A method as in claim 10, wherein the outwardly exposed surface is flattened with rounded corners.

12. A method as in any one of claims 9 to 11, wherein the interface structure comprises a plurality of interface elements.

13. A method as in claim 12, wherein the interface elements are arranged helically over the shell.

14. A method as in any one of claims 9 to 11, wherein the interface structure is elastic so that it closes the shell after expansion.

15. A stent manipulation catheter comprising:
a catheter body having a proximal end and a distal end;
a radially expansible shell near the distal end of the catheter body; and
a stent interface structure circumscribing but not attached to the radially expansible shell.

16. A catheter as in claim 15, wherein said stent interface structure comprises at least one continuous interface element extending over the entire length of the shell.

17. A catheter as in claim 16, wherein at least a portion of said interface element is arranged helically over the shell.

18. A catheter as in claim 15, wherein the expansible shell has an expansible area and the interface structure covers a percentage of the expansible area below 20%.

19. A catheter as in claim 15, wherein at least a portion of the interface structure comprises a wire.

20. A catheter as in claim 15, wherein the interface structure is incorporated in a cage structure which circumscribes the shell.

21. A catheter as in claim 20, wherein the cage structure is attached directly to the catheter body at at least one point.

22. A catheter as in claim 21, further comprising an attachment structure having a proximal end attached to the catheter body and a distal end attached to the cage structure, wherein the attachment structure is sufficiently sized and compliant to accommodate geometrical changes and reaction forces produced by the cage structure as it is expanded by the shell.

23. A catheter as in claim 23, wherein the cage structure is elastic and arranged to radially close over the expansible shell when the shell is collapsed.

24. A catheter as in claim 23, wherein at least a portion of the cage is composed of a superelastic material.

25. A catheter as in claim 15, wherein the assembly of the shell and the interface structure is sufficiently flexible to permit bending at a radius of 10 mm or below when advanced through the coronary vascular.
